(19) United States
(12) Patent Application Publication
(10) Pub. No.: US 2010/0233228 A1
(43) Pub. Date: Sep. 16, 2010

(54) Drug-Eluting Medical Device

(75) Inventor: Ulrich Speck, Berlin (DE)

Correspondence Address: SHOEMAKER AND MATTARE, LTD, 10 POST OFFICE ROAD - SUITE 100, SILVER SPRING, MD, 20910, US

(73) Assignee: INVATEC TECHNOLOGY CENTER GMBH, Frauenfeld (CH)

(21) Appl. No.: 12/721,817

(22) Filed: Mar. 11, 2010

Related U.S. Application Data
(60) Provisional application No. 61/159,507, filed on Mar. 12, 2009.

(51) Int. Cl. A61K 9/00; A61K 31/337; A61P 43/00; A61P 9/08

(52) U.S. Cl. 424/422; 514/449

(57) ABSTRACT

A drug-eluting medical device includes a catheter balloon completely or partially coated with paclitaxel in anhydrous crystalline form, having an immediate release and bioavailability of a therapeutically effective amount of paclitaxel at the intervention site. The balloon can be made of a polyether-polyamide block copolymer, or a polyester amide, or polyamide-12.

This application claims benefit of provisional application 61/159,507, filed Mar. 12, 2009.

### DESCRIPTION

The present invention relates to a drug-eluting medical device, in particular a balloon for angioplasty catheters with drug elution to prevent restenosis of the vessel subjected to angioplasty.

### BACKGROUND OF THE INVENTION

The treatment of vascular atherosclerotic lesions is a widespread therapy. Such lesions are most often localized at predetermined portions of the blood vessels, of which they cause constrictions or also obstructions. Vascular atherosclerotic lesions are typically treated in angioplasty procedures by means of catheters provided with a balloon.

A catheter provided at the distal end thereof with a balloon is advanced, following a guidewire, to the ostium of the narrowed artery. Once the balloon has been arranged at the artery narrowing, it is repeatedly inflated and deflated. The insufflation, with successive deflation, of the balloon within the artery reduce the extent of the arterial luminal narrowing, and restore a suitable blood flow in the cardiac area, suffering from the stenosis. In some cases, it is necessary to arrange a so-called stent, which provides to maintain the artery patent also after withdrawal of the catheter and the balloon.

In both cases, success of the intervention is not complete. In fact, after a few months, some patients develop a new narrowing of the vessel wall at the intervention point. Such narrowing, known under the name of restenosis, is not due to the formation of new atherosclerotic plaques, but to a cell hyperproliferation process, particularly of the vascular smooth muscle cells, probably due to the dilating action operated by the foreign body, stent or balloon.

It has been observed that restenosis can be treated by coating a stent with a drug having antiproliferative action. Among the drugs usually employed to such aim, paclitaxel (taxol) has proved to be particularly efficient. The drug must be released for a sufficiently long time span, so as to inhibit the cell hyper-proliferation process caused by the constant presence of the stent implanted in the vessel. However, the drug also induces an inhibition of the stent endothelization process, which is crucial to avoid the formation of thrombi. For this reasons, the use of a stent with drug elution (“drug eluting stent”) has some drawbacks.

More recently, antiproliferative drug-coated catheter balloons have been proposed. However, in almost all cases, forms of slow release of the drug at the site of intervention after the drug has been transferred from the balloon to the vessel wall have been described.

However, it has been noticed that a drug elution over a prolonged time frame to inhibit the restenosis phenomenon is neither necessary nor desirable, but that it is sufficient, and rather more convenient, a time limited contact between drug and vessel surface, for example, from a few seconds to one minute. These are typically the contact times of a catheter balloon as described before.

The patent publication WO 02/076509 discloses drug-coated catheter balloons releasing such drug in an immediately bioavailable form during the short contact time of the balloon with the vessel wall.

It will be recognized that an approach such as the one described herein above poses completely different problems compared to those previously dealt with. In fact, while a prolonged drug elution can be obtained by various solutions, such as, for example, incorporation of the drug in a polymeric matrix or microcapsules, the immediate release will depend on several factors, of which the main ones are:
The nature of the drug, in particular the hydrophilicity or hydrophobicity thereof;
The form in which the drug is administered, in particular, the crystalline or amorphous form thereof;
The presence of possible excipients or “enhancers”;
Optionally, the nature of the balloon surface on which the drug is deposited.

In fact, it should be understood that the drug has to be, first of all, released from the balloon to the vessel wall in the very short contact time available during an angioplasty procedure. Once the drug has been released, it has to be absorbed by the cell wall, before the blood flow washes it off. Ideally, it is therefore desirable that the drug absorption occurs concomitantly to the release thereof from the balloon.

However, it is just as well necessary that the drug is retained by the balloon surface in a manner sufficient to resist to all the handling operations which it is subjected to, both during the production step and during the preparation and carrying out of the angioplasty procedure, in any case, before the balloon reaches the site of intervention. This requires a perfect balance of such properties.

Therefore, it is an object of the present invention a catheter balloon coated with a drug which allows an immediate release and bioavailability of the drug at the site of intervention.

### SUMMARY OF THE INVENTION

The present invention relates to a catheter balloon coated with paclitaxel in anhydrous crystalline form, having an immediate release and bioavailability of the drug at the intervention site.

According to another aspect of the invention, the catheter balloon coated with paclitaxel in anhydrous crystalline form is made of a polyether-polyamide block copolymer, or “compound” thereof with a polyamide.

According to a further aspect, the catheter balloon coated with paclitaxel in anhydrous crystalline form is made of a polyester amide.

According to a further aspect, the catheter balloon coated with paclitaxel in anhydrous crystalline form is made of polyamide-12.

According to a further aspect, the catheter balloon surface is hydrophilic or made hydrophilic by treatment with a hydrophilizing agent.

According to a further aspect of the invention, paclitaxel in anhydrous crystalline form is deposited from a urea-containing solution.

### DESCRIPTION OF THE INVENTION

The present invention relates in particular to a catheter balloon completely or partially coated with paclitaxel in anhydrous crystalline form, having an immediate release and bioavailability of a therapeutically effective amount of paclitaxel at the intervention site.

By the term “an immediate release and bioavailability” is meant a release from the balloon surface in periods of time ranging between 1 second and 1.5 minutes, preferably between 20 seconds and 1 minute, and an absorption by the vascular tissue in periods of time ranging between 1 second and 25 minutes, preferably between 20 seconds and 25 minutes.

By the term “therapeutically effective amount” is meant a drug amount capable of inducing a therapeutical or preventive effect against the restenosis of the treated vascular tissue in the patient.

By the term “site of intervention” is meant the section of the blood vessel treated directly with the catheter balloon of the invention, and the adjacent portion in the tissues of which the post-procedure presence of paclitaxel can be detected. Generally, such section will extend for 2-10 mm down- and upstream the contact section with the balloon.

By “paclitaxel in anhydrous crystalline form” is meant paclitaxel essentially free from water of crystallization obtained by direct crystallization, or hot and/or vacuum drying, of a hydrated or solvated hydrated form.

This crystalline form of paclitaxel can be obtained by dissolving paclitaxel in an aqueous solvent, by completely or partially wetting the balloon surface with such solution, and by letting the solvent to evaporate, naturally or by hot and/or vacuum drying, to the formation of a crystalline layer having a white, homogeneous, or partially inhomogeneous appearance.

As the aqueous solvent, a mixture of solvents selected from acetone/ethanol/water, tetrahydrofuran/water, methanol/water, acetone/water, ethanol/water, acetonitrile/water, DMF/water is preferably used. More preferably, the solvent is a 9:1 tetrahydrofuran/water mixture or a tetrahydrofuran/water mixture with ratios ranging between 9.5:0.5 and 65:35, or an acetone/ethanol/water mixture in which the organic solvent is present in amounts not less than 50% by volume relative to water.

The balloon wetting step can be performed in several ways, known to those skilled in the art, such as, for example, dipping the balloon into the paclitaxel solution, spraying the paclitaxel solution on the balloon, or depositing the paclitaxel solution on the balloon by means of a syringe, a micropipette, or other similar dispensing device.

The balloon can be wetted with the paclitaxel solution in a deployed and inflated condition, or in a folded condition. It has been observed that in this second case also, the paclitaxel solution penetrates by capillarity under the folds, so as to form a drug depot which remains protected during the introduction step of the folded balloon into the blood vessel by means of the catheter, until reaching the site of intervention and the inflation thereof.

Methods are also known to selectively coat the area under the balloon folds, leaving the outer surface substantially free from the drug. Such methods can comprise, for example, the introduction into the balloon folds of a cannula bearing a series of micro-nozzles, through which the paclitaxel solution is deposited on the inner surface of the folds. Such a method is described, for example, in the international application No. PCT/IT2007/000816, filed on Nov. 21, 2007, the contents of which are incorporated herein by reference.

The folded balloon will preferably have 3 to 6 folds.

A preferred wetting method for the balloon is the deposition of the paclitaxel solution on the folded balloon surface by means of a syringe, micropipette, or other similar dispensing means. Typically, the dispensing means will be made to slide on the surface from an end to the other one, and vice versa, while rotating the balloon around the longitudinal axis thereof, so as to establish a zigzag path. Alternatively, the dispensing means will be made to slide on the balloon surface starting from a substantially central position relative to the longitudinal extent thereof, and it will be made to slide towards a first end thereof and, subsequently, towards the second end thereof, so as to establish a substantially zigzag path.

In general, independently from the method used, it is possible to repeat several times the balloon wetting step with the paclitaxel solution, as a function of the drug amount which is intended to be deposited.

According to a further aspect of the invention, a catheter balloon completely or partially coated with paclitaxel in anhydrous crystalline form, having an immediate release and bioavailability of a therapeutically effective amount of paclitaxel at the intervention site, can be obtained by dissolving paclitaxel in an aqueous solvent, as defined before, in the presence of urea, by completely or partially wetting the balloon surface with such solution, and by letting the solvent to evaporate, naturally or by hot and/or vacuum drying, to the formation of a crystalline layer having a white, homogeneous, or partially inhomogeneous appearance.

It has been noticed that the presence of urea in the coating layer of paclitaxel on the balloon surface promotes the release of the drug from such surface. Urea can be used in amounts ranging between 1 and 100 mg per mL solvent, preferably between 4 and 10 mg per mL solvent, more preferably about 7 mg per mL solvent.

It is a further object of the present invention a catheter balloon completely or partially coated with paclitaxel in anhydrous crystalline form, having an immediate release and bioavailability of a therapeutically effective amount of paclitaxel at the intervention site, in which said balloon is made of a polyether-polyamide block copolymer or “compound” thereof with a polyamide.

The polyether-polyamide block copolymer according to the invention is an elastomer comprising polyamide block-forming monomers, representing the hard portion of the material, modified with a group representing the soft portion.

This elastomer is obtained by polymerization of a polyamide block-forming compound selected from the group consisting of an aminocarboxylic acid according to the formula (1) and a lactam according to the formula (2):

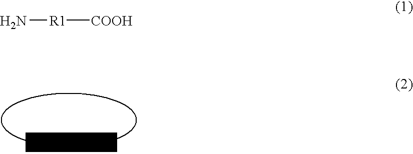

with a triblock polyetherdiamine compound of formula (3):

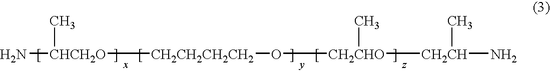

and with a dicarboxylic acid according to the formula (4):

HOOC—(R3)ₘ-COOH (4)

In the above-mentioned formulae, each of the R1, R2, and R3 groups represents linking groups comprising a hydrocarbon chain therein, optionally interrupted by one or more amide groups.

Preferably, R1 and R2 independently comprise an alkylene group having 2 to 20 carbon atoms and amide bonds, and R3 comprises an alkylene group having 1 to 20 carbon atoms;

x can vary between 1 and 20, preferably between 1 and 18, more preferably between 1 and 16; y can vary between 4 and 50, preferably between 5 and 45, more preferably between 8 and 30, and z can vary between 1 and 20, preferably between 1 and 18, more preferably between 1 and 12;

m is 0 or 1.

Generally, the polymerization is carried out by using 15 to 70% by weight of the compound of formula (1) and/or (2), and a mixture of compounds of formulae (3) and (4) in an overall weight percentage between 30 and 85%. This polymerization is carried out in a reactor at a temperature ranging between 150 and 300° C., preferably between 160 and 280° C., more preferably between 180 and 250° C.

Compounds of such copolymers with polyamides can be obtained by mixing, according to known techniques, the copolymer in amounts from 10 to 90% by weight, preferably 75 to 25%, more preferably 60 to 40% by weight, with an amount of polyamide to completion of 100%.

Preferably, the polyamide is polyamide-12.

Such copolymers and the compounds thereof with polyamides are known, and have been described in detail in the patent publication WO 2007/132485 A1, the content of which, relatively to the structure of such materials, and obtaining thereof, is incorporated herein by reference.

It has been observed that the use of such material in the construction of the catheter balloon of the invention provides optimal characteristics of paclitaxel release, while balancing the necessary ability of retaining the drug during the processing and use steps far from the site of intervention with the easiness to release the paclitaxel layer to the vascular cell wall in the short contact time between this and the inflated balloon surface, at the site of intervention.

It is a further object of the present invention a catheter balloon completely or partially coated with paclitaxel in anhydrous crystalline form, having an immediate release and bioavailability of a therapeutically effective amount of paclitaxel at the intervention site, in which said balloon is made of polyamide-12.

It is a further object of the present invention a catheter balloon completely or partially coated with paclitaxel in anhydrous crystalline form, having an immediate release and bioavailability of a therapeutically effective amount of paclitaxel at the intervention site, in which said balloon is made of polyester amide.

The polyester amide used in the present invention can be described by the following general formula:

H—(O—PF—OOC—PA—COO—PF—OOC—PA—CO)ₙ—OH

in which PA is a polyamide segment, PF is a diol segment comprising OH-terminating dimer diol segments, and n is a number ranging between 5 and 20.

The content of the diol component within the polyester-amide copolymer is 5-50% by weight. Preferably, the concentration of diol component ranges between 10 to 30% by weight, still more preferably between 10 and 20% by weight of the total formulation.

These polymers are known, and have been described in detail in the patent publication WO 2005/037337 A1, the content of which, relatively to the chemical structure and the preparation methods of such materials, is incorporated herein by reference.

It is a further object of the present invention a catheter balloon completely or partially coated with paclitaxel in anhydrous crystalline form, having an immediate release and bioavailability of a therapeutically effective amount of paclitaxel at the intervention site, in which said balloon has a surface which is hydrophilic or hydrophilized by suitable hydrophilizing treatment.

For example, the catheter balloon surface according to the invention can be made hydrophilic by treatment with plasma-activated oxygen.

In all the above-described embodiments, paclitaxel is present in the catheter balloon coating layer in amounts ranging between 1 and 20 μg/mm², preferably between 2 and 7 μg/mm², more preferably between 3 and 5 μg/mm².

The invention will now be further described by means of the following examples, given by way of non-limiting example.

### Example 1

### Coating of Catheter Balloons with Crystalline Anhydrous Paclitaxel

Paclitaxel solutions have been prepared at a 50 mg/mL concentration in the following solvents:

(1) 9:1 THF/water

(2) 9:1 THF/water with addition of 15 mg/mL urea

(3) 6.5:3.5 THF/water

(4) Acetone/ethanol/water

(5) Acetic acid (comparative solution)

(6) Dichloromethane (comparative solution)

It shall be noted that paclitaxel in an anhydrous crystalline form according to the invention is not obtained by crystallization from acetic acid. Instead, amorphous paclitaxel is obtained by precipitation from dichloromethane.

Some balloons—made of a polyamide—12+polyether-polyamide block copolymer compound (70% UBESTA® XPA9063+30% UBESTA®-3030XA) and in a folded condition—have been coated with paclitaxel by wetting the surface thereof with equal volumes of the solutions (1)-(6) by means of a Hamilton syringe, according to the previously described modes. For each solution, several balloons have been used.

Then, the balloons have been dried under vacuum.

The appearance of the coating was white, not always homogeneous.

### Example 2

### Assessment of Paclitaxel Adhesion on the Surface of the Catheter Balloons

The balloons prepared according to the example 1 have been subjected to some assessments, in order to determine the drug adhesion under the various conditions.

### Test A

First, the dry adhesion has been assessed, which is useful to determine the paclitaxel loss which can occur in the production or handling steps of the balloon. Such determination has been carried out by dry expanding the balloon and shaking the inflated balloon within a tube.

The paclitaxel content in the tube was determined by HPLC/UV. The drug was taken up with ethanol, the tubes were closed and vigorously vortexed for at least 30 seconds, followed by a treatment in an ultrasound bath for 30 minutes. At least 70 μl of extract were injected into the HPLC, together with a paclitaxel standard solution (concentration of about 20 μg/mL). The results are reported in Table I.

### Test B

Release of paclitaxel at the site of intervention has been assessed in experiments on castrated male pigs, approximately 3 months old, and weighing about 30 kg. The pigs were sedated by intramuscular injection of ketamine and xylazine. Anaesthesia was started by intravenous injection of propofol, followed by orotracheal intubation, and was maintained with 1-2 vol % isoflurane, 70 vol % N₂O₂, and 30 vol % oxygen. All the animals received 5.000 IU heparin, 250 mg aspirine, and 200 mg nitroglicerine via the intracoronary route. The coronary arteries were monitored by means of a standard angiography technique through the left carotid artery.

The animals were treated with the paclitaxel-coated balloons (solutions (1)-(6)) mounted on catheter.

Some balloons, once the site of intervention has been reached, were kept floating in the blood flow for 1 minute without expanding them, then they were retracted, introduced into suitable tubes, inflated, and separated from the catheter. After that, they were extracted with ethanol as described in test A, and finally subjecting the tube to centrifugation for 10 minutes. The extracts were analyzed by HPLC/UV as previously described, so as to determine the paclitaxel amount which is dispersed in the blood flow. The results are reported in Table I.

Other balloons, on which stents had been mounted, have instead been introduced, inflated, and then deflated and retracted, then undergoing the same extraction treatment of the non-inflated ones. In this case, the residual paclitaxel amount left on the balloon after contacting the vessel wall was determined.

After a period of time ranging between 15 and 25 minutes, the animals were sacrificed by administration of 20% KCl under deep anaesthesia. Hearts were quickly removed, and the arterial segments on which the stent was arranged, plus a portion 5 mm down- and upstream the stent, were sectioned, placed in pre-weighted tubes to determine the weight thereof, and subjected to extraction with a predetermined amount of ethanol to achieve a>50% concentration. After 30 minutes of extraction at room temperature with ultrasounds and centrifugation for 10 minutes, the extracts were analyzed by HPLC/UV as described before, so as to determine the paclitaxel amount absorbed by the vascular tissue. The results are reported in Table I.

TABLE I
Results of drug adhesion, release, and uptake by the vascular tissue

| | | % paclitaxel lost in | % paclitaxel not | % paclitaxel |
|---|---|---|---|---|
| Deposition | % paclitaxel lost | blood flow | released to the | absorbed by the |
| solution | by dry expansion | (non-inflated balloon) | intervention site | vascular tissue |
| (1) | 4 ± 3 | 22 ± 3 | 32 ± 9 | 13.3 ± 7.3 |
| (2) | 24 ± 1 | 42 ± 3 | 13 ± 3 | 19.7 ± 11.3 |
| (3) | 10 ± 5 | 26 ± 11 | 30 ± 6 | 17.4 ± 5.5 |
| (4) | 11 ± 11 | 33 ± 13 | 9 ± 4 | 23.4 ± 8.1 |
| (5) | 3 ± 2 | 5 ± 4 | 64 ± 5 | 5.2 ± 3.2 |
| (6) | 4 ± 3 | 41 ± 26 | 11 ± 7 | 17.4 ± 7.2 |

Data reported in Table I show that paclitaxel release is noticeably higher when the drug is present in anhydrous crystalline form (lines (1) to (4)) compared to the hydrated form (line (5)). In fact, in the latter case, most paclitaxel (64%±5%) remains adhered to the balloon surface, and the drug amount absorbed by the vascular tissue is only 5.2%±3.2%.

As regards paclitaxel in the amorphous form (line (6)), although data show a high amount of drug released by the balloon and absorbed into the tissues, further experiments for the restenosis inhibition assessment demonstrated an inactivity of such form. In such further experiments, paclitaxel in anhydrous crystalline form (lines (1)-(4)) exhibited, instead, a restenosis inhibition action in the animal.

Data also show that the presence of urea in the deposition solution (line (2)) produces a higher paclitaxel release and a higher amount of drug absorbed in the vascular tissue, compared to the same solution without the presence of urea (line (1)).

Further investigations demonstrated that the material of which the balloon is made has also a considerable impact on the paclitaxel release properties, the polyether-polyamide block copolymer, or the compound thereof with polyamides giving the best results for drug elution.

### Example 3

### Determination of the Crystalline Form of Paclitaxel

Paclitaxel in anhydrous crystalline form was identified by DSC analysis under the conditions reported in the literature, thus obtaining a profile of thermal events which was equivalent to what has been described in Table I of Jeong Hoon Lee et al., Bull. Korean Chem. Soc. 2001, vol. 22, No. 8, 925-928.

1. A catheter balloon completely or partially coated with paclitaxel in anhydrous crystalline form, having an immediate release and bioavailability of a therapeutically effective amount of paclitaxel at an intervention site.

2. The catheter balloon according to claim 1, wherein said release of a therapeutically effective amount of paclitaxel occurs in between 1 second and 1.5 minutes.

3. The catheter balloon according to claim 1, wherein said bioavailability of a therapeutically effective amount of paclitaxel occurs in between 1 second and 25 minutes.

4. The catheter balloon according to claim 1, wherein said paclitaxel in anhydrous crystalline form is obtained by a method comprising:
dissolving paclitaxel in an aqueous solvent so as to form a paclitaxel solution;
completely or partially wetting the balloon surface with such solution; and
allowing the solvent to evaporate, naturally or by hot and/or vacuum drying.

5. The catheter balloon according to claim 4, wherein said aqueous solvent is selected from acetone/ethanol/water, tetrahydrofuran/water, methanol/water, acetone/water, ethanol/water, acetonitrile/water, DMF/water mixtures.

6. The catheter balloon according to claim 5, wherein said aqueous solvent is selected from a 9:1 tetrahydrofuran/water mixture, a tetrahydrofuran/water mixture with ratios between 9.5:0.5 and 65:35, and an acetone/ethanol/water mixture, in which the organic solvent is present in an amount not less than 50% by volume relative to water.

7. The catheter balloon according to claim 4, wherein said balloon is obtained by depositing said paclitaxel solution on the folded balloon surface by a syringe, micropipette, or other similar dispensing means, and by making said dispensing means to slide on the surface from an end to the other one, and vice versa, while rotating the balloon around the longitudinal axis thereof, so as to establish a zigzag path.

8. The catheter balloon according to claim 4, wherein said paclitaxel solution of the step i) comprises urea, in an amount between 1 and 100 mg/mL.

9. The catheter balloon according to claim 9, wherein said balloon is made of a polyether-polyamide block copolymer, or compound thereof with a polyamide.

10. The catheter balloon according to claim 9, wherein said polyether-polyamide block copolymer is obtained by polymerization of a polyamide block-forming compound selected from the group consisting of an aminocarboxylic acid according to the formula (1) and a lactam according to the formula (2): 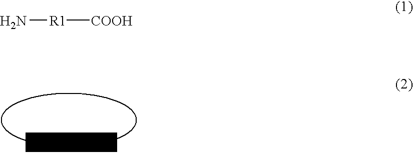
with a triblock polyetherdiamine compound of formula (3): 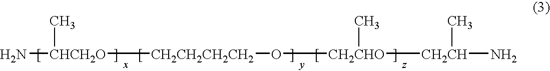
and with a dicarboxylic acid according to the formula (4):
HOOC—(R3)ₘ-COOH (4)
wherein each of the R1, R2, and R3 groups represents linking groups comprising a hydrocarbon chain therein, optionally interrupted by one or more amide groups.

11. The catheter balloon according to claim 10, wherein:
R1 and R2 independently comprise an alkylene group having 2 to 20 carbon atoms and amide bonds;
R3 comprises an alkylene group having 1 to 20 carbon atoms;
x is between 1 and 20, or between 1 and 18, or between 1 and 16;
y is between 4 and 50, or between 5 and 45, or between 8 and 30;
z is between 1 and 20, or between 1 and 18, or between 1 and 12;
m is 0 or 1.

12. The catheter balloon according to claim 10, wherein said polymerization is carried out by using 15 to 70% by weight of the compound of formula (1) and/or (2), and a mixture of compounds of formulae (3) and (4) in an overall weight percentage between 30 and 85%, at a temperature between 150 and 300° C.

13. The catheter balloon according to claim 9, wherein said compounds of the polyether-polyamide block copolymer with a polyamide are obtained by mixing the copolymer in amounts from 10 to 90% by weight, with an amount of polyamide to complete of 100%.

14. The catheter balloon according to claim 13, wherein said polyamide is polyamide-12.

15. The catheter balloon according to claim 1, wherein said balloon is made of polyamide-12.

16. The catheter balloon according to claim 1, wherein said balloon is made of polyester amide.

17. The catheter balloon according to claim 16, wherein said polyester amide is described by the following general formula:
H—(O—PF—OOC—PA—COO—PF—OOC—PA—CO)ₙ—OH
wherein PA is a polyamide segment,
PF is a diol segment comprising OH-terminating dimer diol segments, and
n is a number between 5 and 20.

18. The catheter balloon according to claim 17, wherein the content of the diol component within the polyester-amide copolymer is 5-50% by weight of the total formulation.

19. The catheter balloon according to claim 1, wherein said balloon has a surface which is hydrophilic or hydrophilized by suitable hydrophilizing treatment.

20. The catheter balloon according to claim 1, wherein paclitaxel is present in the catheter balloon coating layer in amounts between 1 and 20 μg/mm².
